SEQUENCE LISTING (2 sequences)

SEQ ID NO: 1 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 2 - protein, 137 aa, Homo sapiens
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

(19) United States
(12) Patent Application Publication
(10) Pub. No.: US 2012/0282654 A1
(43) Pub. Date: Nov. 8, 2012

(54) ANTIBODY PURIFICATION

(75) Inventors: Yan Yao, Chatham, NJ (US); Igor Quinones-Garcia, East Brunswick, NJ (US); Patricia Rowicki, Millburn, NJ (US); John Richard Gavin, Hoboken, NJ (US); Collette Marie Cutler, Bloomingdale, NJ (US); Steven J. Blaisdell, Jackson, NJ (US)

(73) Assignee: Schering Corporation

(21) Appl. No.: 13/266,860

(22) PCT Filed: Apr. 29, 2010
(86) PCT No.: PCT/US10/32898

§ 371 Date: Jul. 2, 2012

Related U.S. Application Data
(60) Provisional application No. 61/173,805, filed on Apr. 29, 2009.
(60) Provisional application No. 61/223,549, filed on Jul. 7, 2009.

(51) Int. Cl. C07K 1/36; C12P 21/02

(52) U.S. Cl. 435/69.6; 530/389.1

(57) ABSTRACT

The present invention provides, in part, a multi-step process for purifying antibodies including chromatographic purification and filter purification.

The present application claims the benefit of U.S. provisional patent application No. 61/173,805; filed Apr. 29, 2009; and, U.S. provisional patent application No. 61/223,549; filed Jul. 7, 2009; each of which is herein incorporated by reference in its entirety.

### FIELD OF THE INVENTION

The field of the present invention relates to methods for purifying proteins such as antibodies.

### BACKGROUND OF THE INVENTION

Purity of the drug substance is the foremost important consideration in the evaluation of a purification process. Purity considerations in monoclonal antibody (mAb) production include the removal of aggregates, host cell proteins (HCP), host cell DNA, leached Protein-A, viruses and other components present in harvested clarified culture medium (HCCM). Purification of mAbs to remove such contaminants is critical to the production of a product which is suitable for human consumption.

### SUMMARY OF THE INVENTION

The present invention provides, in part, a method for purifying an antibody in a composition that specifically binds IGF1R which comprises a CDR-L1, CDR-L2 and CDR-L3 found in a light chain immunoglobulin variable region which comprises the amino acid sequence set forth in SEQ ID NO: 1; and a CDR-H1, CDR-H2 and CDR-H3 found in a heavy chain immunoglobulin variable region which comprises the amino acid sequence set forth in SEQ ID NO: 2; which method comprises (a) purifying the antibody by protein A chromatography; (b) inactivating virus particles in the composition; (c) purifying the antibody by cation-exchange chromatography; (d) purifying the antibody by anion-exchange chromatography; (e) filtering virus particles from the antibody composition; (f) ultrafiltering the antibody composition; (g) diafiltering the antibody composition; and (h) fine filtering the antibody composition. In an embodiment of the invention, the purification is performed between about 20° C. and 25° C. (e.g., 20° C., 21° C., 22° C., 23° C., 24° C., 25° C.). For example, in an embodiment of the invention, protein A chromatography comprises: applying harvested cell culture fluid, comprising the antibody, to a column comprising a protein-A/agarose resin equilibrated with an aqueous solution comprising 10 mM sodium phosphate, 125 mM sodium chloride pH 7.2; wherein the ratio of antibody to volume of resin is about 35 grams/liter; washing the column with an aqueous solution comprising 10 mM sodium phosphate, 125 mM sodium chloride pH 7.2; washing the column with an aqueous solution comprising 10 mM sodium phosphate, pH 7.2; eluting the antibody with 100 mM acetic acid, pH 2.9; and collecting the antibody in the eluate. In an embodiment of the invention, the virus particles are inactivated by adjusting the pH of the antibody composition to about 3.5 for about 1 hour and, the pH is then adjusted to about 5.5 after the virus particles are inactivated. In an embodiment of the invention, the cation-exchange chromatography comprises: applying the antibody to a column comprising sulfopropyl (—CH₂CH₂CH₂SO₃⁻) strong cation-exchange resin equilibrated with an aqueous solution comprising 20 mM sodium acetate, 20 mM sodium chloride, pH 5.5; washing the column with an aqueous solution comprising 20 mM sodium acetate, 20 mM sodium chloride, pH 5.5; eluting the antibody with an aqueous solution comprising 20 mM sodium acetate, 175 mM sodium chloride, pH 5.5; and collecting the antibody in the eluate. In an embodiment of the invention, the anion-exchange chromatography comprises:

applying the antibody to a column comprising strong quaternary ammonium (Q) anion-exchanger resin equilibrated with an aqueous solution comprising 20 mM tris(hydroxymethyl)aminomethane hydrochloride, pH 8.0; washing the column with an aqueous solution comprising 20 mM tris(hydroxymethyl)aminomethane hydrochloride, pH 8.0, wherein the solution washes the unbound antibody through the column; and collecting the antibody in the washed-through eluate. In an embodiment of the invention, ultrafiltering the antibody comprises filtering the antibody containing composition through a membrane under a transmembrane pressure of about 15-25 pounds per square inch (psi) (e.g., 15, 16, 17, 18, 19, 20, 21, 22, 23, 24, 25) by tangential flow wherein the antibody does not pass through the filter and is, instead, retained and concentrated in the retentate. In an embodiment of the invention, diafiltering the antibody comprises filtering the antibody through a membrane under a trans-membrane pressure of about 15-25 pounds per square inch (psi) against about 10 volumes of 5 mM sodium acetate, pH 5.5. In an embodiment of the invention, fine filtering the antibody comprises filtering the antibody through a filter with a pore size of about 0.2 micrometers.

The purification method comprises, in an embodiment, initial application of the antibody, in a harvested clarified culture medium (HCCM), to a column. In an embodiment of the invention, the HCCM is generated by a method comprising:

inoculating an initial mammalian cell growth medium (e.g., comprising HEPES, sodium bicarbonate buffers, inorganic salts, non-essential amino acids, recombinant human insulin, trace elements and surfactants; but not including L-glutamine, antibiotics, antimycotics or animal-derived components) with host cells expressing the antibody and adding supplements comprising: Glucose; L-glutamine; Soy hydrolysate or wheat hydrolysate or both; along with
Adenine sulfate;

### Adenosine;

ammonium vanadate;

### Biotin;

### Choline Chloride;

Cobalt chloride;
Cupric sulfate;

### Cytidine;

### D-Calcium Pantothenate;

### Ethanolamine HCl;

### Flavin Adenine Dinucleotide;

### Folic Acid;

### Glycine;

### Guanosine;

### Hypoxanthine;

i-inositol;

### L-alanine;

### L-arginine;

### L-asparagine;

L-aspartic acid;

### L-citrulline;

### L-cysteine-HCl;

### L-cystine;

L-glutamic acid;

### L-histidine;

### Lipoic Acid;

### L-isoleucine;

### L-leucine;

### L-lysine;

### L-methionine;

### L-ornithine-HCl;

### L-phenylalanine;

### L-proline;

### L-serine;

### L-threonine;

### L-tryptophan;

### L-tyrosine;

### L-valine;

Manganese chloride Tetrahydrate;

### Niacin;

Nickel dichloride hexahydrate;

### Progesterone;

### Putrescine 2HCl;

### Pyridoxine HCl;

### Riboflavin;

Sodium molybdate dehydrate;
Sodium phosphate monobasic;
Sodium selenite;

### Thiamine HCl;

### Thymidine;

Tin chloride dehydrate;

### Uridine;

### Vitamin B12;

### Vitamin E; and

Zinc sulfate;
to the medium; and removing the antibody and culture medium from the host cells. In an embodiment of the invention, the final concentrations of the components added to the medium from the supplements are about those set forth below:

| | | |
|---|---|---|
| Adenine sulfate: | 1.632 | mg/liter |
| Adenosine: | 17.6 | mg/liter |
| Ammonium vanadate: | 0.00078 | mg/liter |
| Biotin: | 0.28 | mg/liter |
| Choline Chloride: | 50.2 | mg/liter |
| Cobalt chloride: | 0.0025 | mg/liter |
| Cupric sulfate: | 0.0032 | mg/liter |
| Cytidine: | 17.6 | mg/liter |
| D-Calcium Pantothenate: | 23.8 | mg/liter |
| Ethanolamine HCl: | 4.4 | mg/liter |
| Flavin Adenine Dinucleotide: | 0.05 | mg/liter |
| Folic Acid: | 4.6 | mg/liter |
| Glycine | 72 | mg/liter |
| Guanosine: | 17.6 | mg/liter |
| Hypoxanthine: | 11.8 | mg/liter |
| i-Inositol: | 73.2 | mg/liter |
| L-alanine: | 8.9 | mg/liter |
| L-arginine | 312.4 | mg/liter |
| L-asparagine: | 842 | mg/liter |
| L-aspartic acid | 97.6 | mg/liter |
| L-citrulline: | 12.6 | mg/liter |
| L-cysteine-HCl | 224 | mg/liter |
| L-cystine: | 34 | mg/liter |
| L-glutamic acid | 155.4 | mg/liter |
| L-histidine | 167 | mg/liter |
| Lipoic Acid: | 0.52 | mg/liter |
| L-isoleucine | 422 | mg/liter |
| L-leucine | 384 | mg/liter |
| L-lysine | 365 | mg/liter |
| L-methionine | 147.2 | mg/liter |
| L-ornithine-HCl: | 25.6 | mg/liter |
| L-phenylalanine | 207 | mg/liter |
| L-proline | 239 | mg/liter |
| L-serine: | 281 | mg/liter |
| L-threonine | 211.6 | mg/liter |
| L-tryptophan | 109.2 | mg/liter |
| L-tyrosine | 234 | mg/liter |
| L-valine | 308.8 | mg/liter |
| Manganese chloride tetrahydrate: | 0.0003 | mg/liter |
| Niacin: | 31.4 | mg/liter |
| Nickel dichloride hexahydrate: | 0.0004 | mg/liter |
| Progesterone: | 0.015 | mg/liter |
| Putrescine 2HCl: | 0.4 | mg/liter |
| Pyridoxine HCl: | 3 | mg/liter |
| Riboflavin: | 1.86 | mg/liter |
| Sodium molybdate dehydrate: | 0.00016 | mg/liter |
| Sodium phosphate monobasic: | 288.2 | mg/liter |
| Sodium selenite: | 0.01426 | mg/liter |
| Thiamine HCl: | 16 | mg/liter |
| Thymidine: | 7.8 | mg/liter |
| Tin chloride dehydrate: | 0.00008 | mg/liter |
| Uridine: | 17.6 | mg/liter |
| Vitamin B12: | 3.4 | mg/liter |
| Vitamin E: | 0.376 | mg/liter |
| Zinc sulfate: | 1.08 | mg/liter |
| Glucose | 1.5 | g/liter |
| L-glutamine | 150 | mg/liter |

In an embodiment of the invention, the supplements are added from an amino acid feed that comprises amino acids at about the following concentrations:
L-arginine: 6.32 g/liter
L-cystine: 1.7 g/liter
L-histidine: 2.1 g/liter
L-isoleucine: 2.6 g/liter
L-leucine: 2.6 g/liter
L-lysine: 3.6 g/liter
L-Methionine: 0.76 g/liter
L-phenylalanine: 1.65 g/liter
L-threonine: 2.38 g/liter
L-tryptophan: 0.51 g/liter
L-tyrosine: 1.8 g/liter
L-valine: 2.34 g/liter;
for example, wherein about 20 ml of this amino acid feed is added per liter of culture medium. In an embodiment of the invention, the supplements are added from an amino acid feed that comprises amino acids at about the following concentrations:
L-alanine: 0.89 g/liter
L-asparagine: 1.5 g/liter
L-aspartic acid: 1.33 g/liter
L-glutamic acid: 1.47 g/liter
Glycine: 0.75 g/liter
L-proline: 1.15 g/liter
L-serine: 1.05 g/liter;
for example, wherein about 10 ml of this amino acid feed is added per liter of culture medium. In an embodiment of the invention, the supplements are added from a nutrient feed that comprises supplements at about the following concentrations:

| | | |
|---|---|---|
| L-asparagine: | 40.6 | g/liter |
| L-proline | 10.81 | g/liter |
| L-isoleucine | 18.53 | g/liter |
| L-cysteine-HCl | 11.19 | g/liter |
| L-leucine | 16.58 | g/liter |
| L-threonine | 8.2 | g/liter |
| L-tyrosine | 9.9 | g/liter |
| L-arginine | 9.29 | g/liter |
| L-aspartic acid | 3.56 | g/liter |
| L-glutamic acid | 6.28 | g/liter |
| Glycine | 2.83 | g/liter |
| L-histidine | 6.23 | g/liter |
| L-methionine | 6.58 | g/liter |
| L-tryptophan | 4.93 | g/liter |
| L-lysine | 14.66 | g/liter |
| L-phenylalanine | 8.64 | g/liter |
| L-valine | 13.08 | g/liter |
| L-serine: | 13 | g/liter |
| Sodium phosphate Monobasic: | 14.41 | g/liter |
| Zinc sulfate: | 0.054 | g/liter |
| Cupric sulfate: | 0.00016 | g/liter |
| Ammonium vanadate: | 0.000039 | g/liter |
| Cobalt chloride: | 0.000125 | g/liter |
| Nickel dichloride Hexahydrate: | 0.00002 | g/liter |
| Sodium molybdate dehydrate: | 0.000008 | g/liter |
| Tin chloride dehydrate: | 0.000004 | g/liter |
| Manganese chloride: tetrahydrate: | 0.000015 | g/liter.; |

for example, wherein about 20 ml nutrient feed is added per liter of culture medium. In an embodiment of the invention, the supplements are added from a vitamin/salt feed that comprises supplements at about the following concentrations:
Sodium selenite: 7.13×10⁻⁴ g/liter
Adenine sulfate: 0.0816 g/liter
Adenosine: 0.88 g/liter
Cytidine: 0.88 g/liter
Guanosine: 0.88 g/liter
Uridine: 0.88 g/liter
Hypoxanthine: 0.59 g/liter
L-citrulline: 0.63 g/liter
L-ornithine-HCl: 1.28 g/liter
Biotin: 0.014 g/liter
Flavin Adenine Dinucleotide: 0.0025 g/liter
Folic Acid: 0.23 g/liter
Lipoic Acid: 0.026 g/liter
Niacin: 1.57 g/liter
Pyridoxine HCl: 0.15 g/liter
Riboflavin: 0.093 g/liter
Thiamine HCl: 0.8 g/liter
Vitamin E: 0.0188 g/liter
Vitamin B12: 0.17 g/liter
Choline Chloride: 2.51 g/liter
Ethanolamine HCl: 0.22 g/liter
i-Inositol: 3.66 g/liter
Thymidine: 0.39 g/liter
Putrescine 2HCl: 0.02 g/liter
Progesterone: 0.00075 g/liter
D-Calcium Pantothenate: 1.19 g/liter;
for example, wherein about 20 ml vitamin/salt feed is added per liter of culture medium. In an embodiment of the invention, in generating the HCCM, the culture medium is harvested from the host cells when viability of the cells is below about 60%. In an embodiment of the invention, the method further comprises purifying the antibody and culture medium from the cells by centrifuging the antibody and culture medium and/or depth filtering the antibody and culture medium and/or filtering the antibody and culture medium through a 0.2 micron filter. In an embodiment of the invention, the purification method of the invention includes the steps of inoculating an initial mammalian cell growth medium, pre-warmed to about 37° C.; which initial medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing the antibody light chain immunoglobulin and heavy chain immunoglobulin, to a cell density of about 2.5−5×10⁵ cells/ml (e.g., 2.5×10⁵, 2.7×10⁵, 2.9×10⁵, 3×10⁵, 3.5×10⁵, 4×10⁵, 4.5×10⁵, 5×10⁵); and, adding the following supplements to the medium before, simultaneously with or immediately after said inoculation: soy hydrolysate to a final concentration of about 10 g/liter; and, optionally, an amino acid feed wherein the concentrations of the components added by said amino acid feed are approximately those set forth below:
L-arginine: 126.4 mg/liter
L-cystine: 34 mg/liter
L-histidine: 42 mg/liter
L-isoleucine: 52 mg/liter
L-leucine: 52 mg/liter
L-lysine: 72 mg/liter
L-Methionine: 15.2 mg/liter
L-phenylalanine: 33 mg/liter
L-threonine: 47.6 mg/liter
L-tryptophan: 10.2 mg/liter
L-tyrosine: 36 mg/liter
L-valine: 46.8 mg/liter
L-alanine: 8.9 mg/liter
L-asparagine: 30 mg/liter
L-aspartic acid: 26.6 mg/liter
L-glutamic acid: 29.4 mg/liter
glycine: 15 mg/liter
L-proline: 23 mg/liter
L-serine: 21 mg/liter;
and, when viable cell density reaches over about 1.2×10⁶ cells/ml, adding supplement feeds wherein the concentrations of the components added by said supplement feeds are approximately those set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, maintaining glucose concentration in the medium at about 1.5 g/liter and maintaining L-glutamine concentration in the medium at about 150 mg/liter; and during cell growth maintaining O₂ concentration at about 60%; pH at about 6.8±0.02 and temperature at about 36.5° C.±0.5° C.; and, optionally, removing the host cells from the medium when cell viability is below about 60%. In an embodiment of the invention, removing the host cells from the medium comprises recovering the culture medium from the cells by disk-stack centrifuging the medium, depth filtering the medium and filtering the medium through a filter with about a 0.2 micron pore size.

### BRIEF DESCRIPTION OF THE FIGURES

FIG. 1. Flow diagram for an embodiment of the anti-IGF1R antibody purification process of the present invention.

FIG. 2. Monomer/aggregate separation performance of several cation-exchange columns.

### DETAILED DESCRIPTION OF THE INVENTION

The present invention provides a purification process that has been extensively researched and customized to purify the anti-IGF1R antibody of the present invention (comprising amino acids 20-128 of SEQ ID NO: 1 and amino acids 20-137 of SEQ ID NO: 2 (gamma-1/kappa)) to a superior level of purity. The cation-exchange purification conditions of the process have been especially fine-tuned to match the particular requirements of the anti-IGF1R antibody so as to achieve exceptional purity.

The process for the purification of anti-IGF1R includes the following unit operations: protein A chromatography, viral inactivation, cation-exchange chromatography, anion-exchange chromatography, virus filtration, ultrafiltration/diafiltration (UF/DF) and final filtration. The purification procedure can be carried out at room temperature (20-25° C.). The process of the present invention delivers good quality antibody and removes aggregates, leached protein A, viruses, endotoxin, host cell proteins and DNA. At the same time, the process delivers appropriate control of bioburden in the process intermediates and purified antibody.

### Protein-A Chromatography

“Protein A” encompasses Protein A recovered from a native source or produced synthetically (e.g., by peptide synthesis or by recombinant techniques) along with functional variants thereof which retain the ability to bind immunoglobulin-gamma (IgG) proteins (e.g., human IgG such as IgG1, IgG2 or IgG4), e.g., the C_{H}2/C_{H}3 region thereof. Protein A and chromatographic resins comprising protein-A can be purchased commercially.

For use in chromatography, Protein A can be immobilized on a solid phase. A solid phase includes an insoluble matrix to which the Protein A is tethered. Solid phases may be, for example, agarose, polystyrene, latex, sepharose (e.g., sepharose 4B), glass or silica. In certain embodiments, the solid phase is coated with a reagent (such as glycerol) which is intended to prevent nonspecific adherence of contaminants to the solid phase.

### Anion and Cation-Exchange Chromatography

The charge characteristics and molecular size of the protein, and the physicochemical properties of the resin are important factors that determine the ion exchange chromatography behavior, especially for macromolecules, such as mAbs (monoclonal antibodies), the size of which are similar to those of the pore structure in most resins for bioseparation.

Optimal pH and ionic strength for appropriate binding is a prerequisite for separation in cation-exchange chromatography, which will be determined with the consideration of good binding and antibody stability. For example, in an embodiment of the invention, a cation-exchange resin, such as a sulfopropyl cation-exchanger, is equilibrated and/or washed, following the sample, at a pH of about 5.5, for example in a buffer comprising about 20 mM acetate (e.g., sodium acetate) and about 20 mM salt (e.g., NaCl). Typical elution patterns of antibody and impurities in bind-and-elute cation-exchange chromatography are as follows: a fraction of host cell proteins flow through or co-elute with the antibody, and protein-A and the majority of host cell proteins elute during the regeneration step. Aggregates tend to bind stronger than monomer, and elute in the peak tail or during the regeneration step. Retention extent in ion exchange chromatography is closely related to the ionic strength. Lower NaCl concentration during elution can help improve the separation resolution due to less screened electrostatic interactions, which maximize the retention differences between monomer and the more strongly bound impurities, including aggregates. However, when the NaCl concentration is too low, strong antibody-resin interactions will result in large pools and/or low recovery. So the focus of the design of an elution step can be the elution and pooling strategy to achieve a good balance among purity, yield and pool size. Elution can be carried out via a linear or step gradient. Stepwise elution chromatography is commonly employed in process chromatography for simplicity of implementation and higher pool concentration. In an embodiment of the invention, the antibody is eluted in higher salt than the wash, e.g., with an elution buffer comprising a pH of about 5.5, about 20 mM acetate (sodium acetate) and about 175 mM salt (e.g., NaCl).

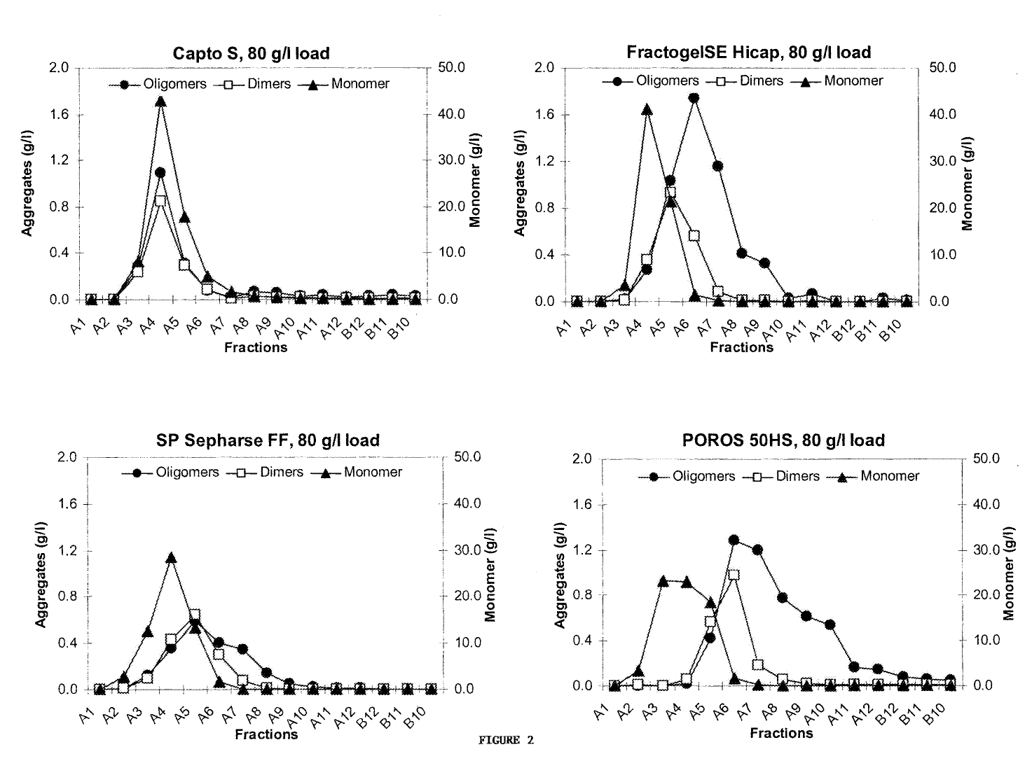

Cation-exchange conditions that reach optimum purity for one antibody may not, necessarily, reach an optimal level of purity when used in connection with a different antibody. In developing the purification process of the present invention, cation-exchange conditions were tailored to the particular characteristics of the anti-IGF1R antibody of the present invention (comprising amino acids 20-128 of SEQ ID NO: 1 and amino acids 20-137 of SEQ ID NO: 2). For example, the cation-exchange conditions reached were highly efficient at removing aggregates. Optimization included screening several different cation exchange resins and development of the elution strategy for the most optimal impurity clearance, production rate, step recovery and pool size. The ionic strength of the elution buffer was optimized in order to enable the best separation of aggregates and monomer. The pooling criteria were established by excluding from the pool those fractions with higher levels of aggregates. A step-gradient elution procedure for the cation-exchange resin that was ultimately chosen for the process was modulated and tested to arrive at the most optimal operating conditions. FIG. 2 sets forth the separation profiles of aggregates and monomer obtained with four of the resins tested at a fairly high column load (˜80 grams/liter). Clearly, the POROS 50HS provided the greatest level of resolution between oligomers, dimers and monomers. Furthermore, Table 9 summarizes the purification performance of several resins at their optimal elution conditions, further confirming that POROS 50 HS provided the best compromise between related purity, adequate step yield, acceptable pool size and increased production rate (e.g., higher flow rate).

The pH and conductivity of the feed for the anion-exchange chromatography are critical parameters for clearance effectiveness by modulating the electrostatic interactions, with higher pH and lower conductivity yielding better impurity reduction in mAb purification. Typically, the pH and conductivity of the feed are chosen to be 0.5-1 unit below the antibody pI and <7.5 mS/cm (millisiemens/centimeter), respectively. The pI of the antibody of the present invention is 8.59, so the anion-exchange conditions of the feed were set to pH 8.0.

A relatively high column load in terms of product in flow-through anion-exchange chromatography should be feasible, as trace-amounts of impurities, rather than the product, are retained. Despite the high column load attainable, it would, in one embodiment of the invention, be required to use a production-scale anion-exchange column that is oversized in order to obtain good throughput considering the limited permeability of a number of commonly used anion-exchangers which exhibit significant compressability. The over-sizing strategy may not, in an embodiment of the invention, be desirable as it requires more resin and takes up a large footprint in manufacturing facilities. This is addressed in the present invention by the use of a more rigid anion exchange resin.

In an embodiment of the invention, an anion-exchange column (e.g., quaternary ammonium) is equilibrated and washed with a buffer at a pH of about 8.0 and 20 mM tris (hydroxymethyl)aminomethane hydrochloride. Following elution of the antibody from the column, the pH can then, in an embodiment of the invention, be adjusted to about 5.5 (e.g., 100 mM acetic acid, pH 5.5)

Anion-exchange resins include those with quaternary ammonium anion-exchangers. Cation-exchange resins include those with sulfopropyl (—CH₂CH₂CH₂SO₃⁻) cation-exchangers.

### Filtration

Besides chromatography, membrane filtration is another integral technique for the recovery and purification of biopharmaceuticals such as therapeutic antibodies. The filtration techniques frequently used in bioseparation include depth filtration for secondary clarification of centrifuged cell culture harvest; microfiltration of in-process intermediate for particulate removal and bioburden control; virus filtration to ensure viral clearance, and ultrafiltration and diafiltration (UF/DF) for buffer exchange and concentration of the product (discussed infra).

Optimized filter sizing has great economic significance considering the high cost of the single-use virus filters. The sizing strategy should be guided by the filter capacity for effective viral clearance, and the tradeoff between filtration time and filter cost. The performance aspects relevant to filter sizing include flux decay during the filtration of a representative feed stream, and the maximum volume of feed that can be filtered before deterioration of virus clearance effectiveness. Validation of a virus clearance step usually involves scale-down models to evaluate the clearance of spiked-in model viruses. Typically an LRV (logarithmic reduction value) of greater than 4 is considered effective for a viral clearance step.

“Pore size” with regard to a filter refers to the average diameter of the pores in the filter.

### Ultrafiltration and Diafiltration

Ultrafiltration (UF) is a variety of membrane filtration in which hydrostatic pressure forces a liquid against a semipermeable membrane. Suspended solids and solutes of high molecular weight are retained, while water and low molecular weight solutes pass through the membrane. Typically, a sample is placed in a device containing a suitable ultrafiltration membrane that will retain the antibodies. Pressure is applied as volume passes through the membrane. The large antibody molecules are retained in the retentate. The filtrate contains low molecular weight components (e.g., salt and water) but little or none of the large antibody molecules. Therefore, the antibodies are concentrated as liquid and salt are removed. Generally, the low molecular weight composition in the concentrate remains constant so the ionic strength of the concentrated solution remains relatively constant.

Diafiltration typically uses ultrafiltration membranes in a similar manner as ultrafiltration; except that this technique can be used to remove, replace, or lower the concentrations of salts or buffering components from solutions containing proteins, such as antibodies, peptides, nucleic acids, and other biomolecules. Continuous diafiltration (also referred to as constant volume diafiltration) involves washing out the original buffer salts (or other low molecular weight species) in the retentate (sample) by adding water or a new buffer to the retentate, e.g., at the same rate as filtrate is being generated. In an embodiment of the invention, and as a result, the retentate volume and product concentration does not change appreciably during the diafiltration process. If water is used for diafiltering, the salts will be washed out and the conductivity lowered. If a buffer is used for diafiltering, the new buffer salt concentration will increase at a rate inversely proportional to that of the species being removed. The amount of salt removed is related to the filtrate volume generated, relative to the retentate volume. The filtrate volume generated is usually referred to in terms of “diafiltration volumes”. A single diafiltration volume (DV) is the volume of retentate when diafiltration is started. When the volume of filtrate collected equals the starting retentate volume, 1 DV has been processed. In general, using continuous diafiltration, greater than 99.5% of a 100% permeable solute can be removed by washing through about 6 retentate volumes (6 DV) with the buffer of choice.

Typically, discontinuous diafiltration by sequential dilution involves first diluting the sample with water or replacement buffer to a predetermined volume. The diluted sample is then concentrated back to its original volume. This process is repeated until the unwanted salts, solvents, or smaller molecules are removed. Each subsequent dilution removes more of the small molecules.

Typically, discontinuous diafiltration by volume reduction reverses this procedure. The sample is first concentrated, to a predetermined volume, and then diluted back to its original volume with water or replacement buffer. This is repeated until the unwanted salts, solvents, or smaller molecules are removed. Each subsequent concentration and dilution removes more of the small molecule.

### Clarified Culture Medium

Clarified culture medium for use in the purification process of the invention can be generated by any conventional method. In an embodiment of the invention, cells expressing and secreting the antibody to be purified are grown and the culture is harvested for further processing as set forth herein. Two processes are described herein: process #1 and #2.

### Process #1

Cells are initially inoculated at 3-4×10⁵ cells/ml in the EX-CELL ACF CHO medium (Sigma-Aldrich; St. Louis, Mo.) which is pre-warmed to 37° C. and adjusted to pH 6.8. The feeds added in some embodiments of process #1 are summarized below in Table 1.

TABLE 1
Feeds added for process #1 runs.

| | | | Volume | |
|---|---|---|---|---|
| | | | ratio | |
| | | | (volume | In-process |
| | | Time | feed/volume | temperature |
| Batch | Feeds added | added | batch) | downshift |
| 1 | SHYS feed | Day 0 | 0.05 | Yes: day 6 |
| | CHO feed1 | Day 3 | 0.02 | from 37° C. to |
| | CHO feed2 | Day 3 | 0.02 | 34° C. |
| 2 | SHYS feed | Day 0 | 0.05 | Yes: day 6 |
| | CHO feed1 | Day 3 | 0.02 | from 37° C. to |
| | CHO feed2 | Day 3 | 0.02 | 34° C. |
| 3 | HYS feed | Day 0 | 0.05 | Yes: day 6 |
| | CHO feed1 | Day 3 | 0.02 | from 37° C. to |
| | CHO feed2 | Day 3 | 0.02 | 34° C. |
| 4 | HYS feed | Day 0 | 0.05 | Yes: day 6 |
| | CHO feed1 | Day 3 | 0.02 | from 37° C. to |
| | CHO feed2 | Day 3 | 0.02 | 34° C. |
| 5 | SHYS feed | Day 0 | 0.05 | No |
| | 50X amino acid feed | Day 0 | 0.02 | |
| | 100X amino acid | Day 0 | 0.01 | |
| | feed | | | |
| | CHO feed1 | Day 3 | 0.02 | |
| | CHO feed2 | Day 3 | 0.02 | |

SHYS feed: a 200 g/L (aq) soy hydrolysate feed from DMV international (Netherlands).
Hys feed: a 200 g/L (aq) soy hydrolysate feed from Kerry Biosciences.
CHO feed 1: 50X Vitamin/salt feed.
CHO feed 2: 50X Nutrient feed
50X amino acid feed.
100X amino acid feed.
pH is continuously monitored and adjusted to a setpoint of 6.8.
Oxygen concentration is continuously monitored and adjusted to a setpoint of 60%.
Temperature is continuously monitored and maintained at 37 ± 1° C. An in-process temperature downshift to 34° C. is performed in the indicated batches.

Glucose is added, for example, when the glucose concentration in the culture medium falls below 1.5 g/liter and L-glutamine is added, for example, when the glutamine concentration in the culture medium falls below 150 mg/liter.

In an embodiment of the invention, the osmolality is shifted to over 400 mOsm from addition of the nutrient feed.

In an embodiment of the invention, the cells are harvested between days 21-24 or, in an embodiment of the invention, earlier (e.g., between days 14-18); generally, when cell viability is reduced to about 60%.

The addition of amino acid feeds may be omitted when the nutrient feeds are used in process #1.

### Process #2

Cells are initially inoculated at 3-4×10⁵ cells/ml in the EX-CELL ACF CHO medium (Sigma-Aldrich; St. Louis, Mo.) which is pre-warmed to 37° C. and adjusted to pH 6.8. The feeds added in some embodiments of process #2 are summarized below in Table 2,

TABLE 2
Feeds added for process #2 runs

| | | | Volume ratio | |
|---|---|---|---|---|
| | | | (volume | In-process |
| | | Time | feed/volume | temperature |
| Batch | Feeds added | added | batch) | downshift |
| A | SHYS feed | Day 0 | 0.05 | No |
| | 50X amino acid feed | Day 0 | 0.02 | |
| | 100X amino acid | Day 0 | 0.01 | |
| | feed | | | |
| | CHO feed1 | Day 3 | 0.02 | |
| B | SHYS feed | Day 0 | 0.05 | No |
| | 50X amino acid feed | Day 0 | 0.02 | |
| | 100X amino acid | Day 0 | 0.01 | |
| | feed | | | |
| | CHO feed1 | Day 3 | 0.02 | |

SHYS feed: a 200 g/L (aq) soy hydrolysate feed from DMV international (Netherlands).
CHO feed1: 50X Vitamin/salt feed.
50X amino acid feed.
100X amino acid feed.
pH is continuously monitored and maintained at 6.8 ± 0.02.
Oxygen concentration is continuously monitored and adjusted to a setpoint of 60%.
Temperature is continuously monitored and maintained at 36.5 ± 0.5° C.

Glucose is added, for example, when the glucose concentration in the culture medium falls below 1.5 g/liter and L-glutamine was added, for example, when the glutamine concentration in the culture medium falls below 150 mg/liter.

The feeds used and the final concentrations of the components of each feed are set forth below.

TABLE 3
Vitamin/salt feed

| | Concentration in | Final concentration in |
|---|---|---|
| Component | feed (g/L) | culture (mg/L) |
| Sodium selenite | 7.13 × 10⁻⁴ | 0.01426 |
| Adenine sulfate | 0.0816 | 1.632 |
| Adenosine | 0.88 | 17.6 |
| Cytidine | 0.88 | 17.6 |
| Guanosine | 0.88 | 17.6 |
| Uridine | 0.88 | 17.6 |
| Hypoxanthine | 0.59 | 11.8 |
| L-citrulline | 0.63 | 12.6 |
| L-ornithine-HCl | 1.28 | 25.6 |
| Biotin | 0.014 | 0.28 |
| Flavin Adenine Dinucleotide | 0.0025 | 0.05 |
| Folic Acid | 0.23 | 4.6 |
| Lipoic Acid | 0.026 | 0.52 |
| Niacin | 1.57 | 31.4 |
| Pyridoxine HCl | 0.15 | 3 |
| Riboflavin | 0.093 | 1.86 |
| Thiamine HCl | 0.8 | 16 |
| Vitamin E | 0.0188 | 0.376 |
| Vitamin B12 | 0.17 | 3.4 |
| Choline Chloride | 2.51 | 50.2 |
| Ethanolamine HCl | 0.22 | 4.4 |
| i-Inositol | 3.66 | 73.2 |
| Thymidine | 0.39 | 7.8 |
| Putrescine 2HCl | 0.02 | 0.4 |
| Progesterone | 0.00075 | 0.015 |
| D-Calcium Pantothenate | 1.19 | 23.8 |

TABLE 4
Amino acid feed #1 (50X).

| | Concentration in feed | Final concentration in culture |
|---|---|---|
| Component | (g/L) | (mg/L) |
| L-arginine | 6.32 | 126.4 |
| L-cystine | 1.7 | 34 |
| L-histidine | 2.1 | 42 |
| L-isoleucine | 2.6 | 52 |
| L-leucine | 2.6 | 52 |
| L-lysine | 3.6 | 72 |
| L-Methionine | 0.76 | 15.2 |
| L-phenylalanine | 1.65 | 33 |
| L-threonine | 2.38 | 47.6 |
| L-tryptophan | 0.51 | 10.2 |
| L-tyrosine | 1.8 | 36 |
| L-valine | 2.34 | 46.8 |

TABLE 5
Amino acid feed #2 (100X).

| | Concentration in feed | Final concentration in culture |
|---|---|---|
| Component | (g/L) | (mg/L) |
| L-alanine | 0.89 | 8.9 |
| L-asparagine | 1.5 | 30 |
| L-aspartic acid | 1.33 | 26.6 |
| L-glutamic acid | 1.47 | 29.4 |
| Glycine | 0.75 | 15 |
| L-proline | 1.15 | 23 |
| L-serine | 1.05 | 21 |

TABLE 6
Nutrient feed.

| | Concentration | Final concentration in |
|---|---|---|
| Component | in feed (g/L) | culture (mg/L) |
| L-asparagine | 40.6 | 812 |
| L-serine | 13 | 260 |
| L-proline | 10.81 | 216 |
| L-isoleucine | 18.53 | 370 |
| L-cysteine-HCl | 11.19 | 224 |
| L-leucine | 16.58 | 332 |
| L-threonine | 8.2 | 164 |
| L-tyrosine | 9.9 | 198 |
| L-arginine | 9.29 | 186 |
| L-aspartic acid | 3.56 | 71 |
| L-glutamic acid | 6.28 | 126 |
| glycine | 2.83 | 57 |
| L-histidine | 6.23 | 125 |
| L-methionine | 6.58 | 132 |
| L-tryptophan | 4.93 | 99 |
| L-lysine | 14.66 | 293 |
| L-phenylalanine | 8.64 | 174 |
| L-valine | 13.08 | 262 |
| Sodium phosphate monobasic | 14.41 | 288.2 |
| Zinc sulfate | 0.054 | 1.08 |
| Cupric sulfate | 0.00016 | 0.0032 |
| Ammonium vanadate | 0.000039 | 0.00078 |
| Cobalt chloride | 0.000125 | 0.0025 |
| Nickel dichloride hexahydrate | 0.00002 | 0.0004 |
| Sodium molybdate dihydrate | 0.000008 | 0.00016 |
| Tin chloride dihydrate | 0.000004 | 0.00008 |
| Manganese chloride tetrahydrate | 0.000015 | 0.0003 |

Following growth, in an embodiment of the invention, the cell culture is recovered from the cells, e.g., by disk-stack centrifuging the medium, depth filtering the medium and filtering the medium through a filter with a 0.2 micron pore size to generate a harvested clarified culture fluid (HCCF) or harvested clarified culture medium (HCCM) for purification using the method of the present invention.

In an embodiment of the invention, the initial mammalian cell growth medium to which the supplements are added comprises HEPES, sodium bicarbonate buffers, inorganic salts, non-essential amino acids, recombinant human insulin, trace elements and surfactants; and does not comprise L-glutamine, antibiotics, antimycotics or animal-derived components.

Generally, for the purposes of the present invention, a “hydrolysate feed” includes wheat and/or soy hydrolysates. Generally, a soy or wheat hydrolysate is the product of an enzymatic digest of soy or wheat and can be purchased commercially. Typically, the hydrolysate is in cell culture grade water and is sterile. In an embodiment of the invention, the hydrolysate is a stock solution at 200 g/liter. In an embodiment of the invention, the hydrolysate is added to the culture medium to reach a final concentration of about 10 g/liter. In an embodiment of the invention, when using either process #1 or #2, the hydrolysate is added to the culture medium either initially, before, with or immediately after inoculation or at about 3 days after inoculation or when viable cell density reaches over about 1×10⁶ cells/ml.

“Viable cell density” refers to the concentration of cells in the medium being analyzed (e.g., cells/ml) which are viable, e.g., capable of growth and replication (e.g., when used to inoculate a liquid culture or a solid culture medium) or capable of excluding a dye such as tryptan blue, eosin or propidium in a dye exclusion assay. Such assays are commonly known in the art.

In an embodiment of the invention, the vitamin/salt feed is a 50× stock solution. In an embodiment of the invention, the vitamin/salt feed is added to the culture medium to reach a final concentration of about 20 ml/liter. When employing process #2, the vitamin/salt feed is added to the culture between days 3 and 5, post-inoculation, or when viable cell density reaches over about 1×10⁶ cells/ml. In an embodiment of the invention, when employing process #1, the vitamin/salt feed is added to the culture between days 3 and 5, post-inoculation, or when viable cell density reaches over about 1.2×10⁶ cells/ml.

In an embodiment of the invention, two separate amino acid feed stock solutions are prepared: a 100× stock solution including L-alanine, L-asparagine, L-aspartic acid, L-glutamic acid, glycine, L-proline and L-serine at the concentrations set forth above; and and a 50× solution including L-arginine, L-cystine, L-histidine, L-isoleucine, L-leucine, L-lysine, L-Methionine, L-phenylalanine, L-threonine, L-tryptophan, L-tyrosine, and L-valine at the concentrations set forth above. These stocks can be made and added separately to the culture medium. In an embodiment of the invention, the amino acid stock solution is added to the initial medium at day 0, before, with or immediately after cell inoculation.

In an embodiment of the invention, the nutrient feed is a 50× stock solution. In an embodiment of the invention, the nutrient feed is added to the culture medium to reach a final concentration of about 20 ml/liter. When employing process #1, in an embodiment of the invention, the nutrient feed is added to the culture between days 3 and 5, post-inoculation, or when viable cell density reaches about 1.2×10⁶ cells/ml.

Furthermore, in an embodiment of the invention, when employing either process #1 or #2, glucose (from a 2.5 M stock solution) and L-glutamine (from a 0.2 M stock solution) are added to the culture medium at any point, e.g., when the concentrations of the nutrients fall below 1.5 g/liter glucose and 150 mg/liter L-glutamine.

Host cells that can be used to express an antibody include mammalian cells, for example, Chinese hamster ovary cells (CHO cells). A CHO-K1 cell is proline-requiring and is diploid for the dihydrofolate reductase (dhfr) gene; in an embodiment of the invention, the host cell is a CHO-K1 cell. In an embodiment of the invention, the cell line is the DXB11 CHO cell line (Urlaub et al. (1983) Cell 33: 405-412). Other cell lines include, for example, HEK293.

### Purification Process

In an embodiment of the invention, the antibody prepared is initially expressed recombinantly in a Chinese hamster ovary (CHO) cell culture, wherein the antibody is secreted from the cells, into the culture medium; wherein the CHO cells are removed from the culture medium to generate a harvested clarified cell culture fluid (HCCF) (e.g., as set forth herein).

The present invention includes a purification process comprising the steps below. Embodiments of the invention include those wherein one or more steps in the process (e.g., 1-7) are modified with one or more of the conditions/parameters (in whole or in part) associated with the step as set forth in the description below. In an embodiment of the invention, the purification process comprises the steps of:

(1) purifying the clarified cell culture fluid comprising the antibody on a protein A (e.g., protein A/agarose) chromatography column:
e.g., wherein the column is packed at bed height of about 20 cm; and/or
e.g., wherein the bed is sanitized at about 3 cm/min with about 3 bed volumes (BV) of solution J4 (0.1N sodium hydroxide, 1M sodium chloride); and/or
e.g., wherein the column is regenerated upflow at about 3 cm/min with about 3 BV of solution J3 (100 mM acetic acid, pH approximately 2.9); and/or
e.g., wherein the column is equilibrated with solution J1, for example, downflow at about 6 cm/min with about 5 BV of solution J1 (pH 7.2, 10 mM sodium phosphate, 125 mM sodium chloride); and/or
e.g., wherein the antibody (e.g., HCCF) is loaded on the column, for example, wherein the load of antibody per volume of packed resin is equal to or less than about 35 g/L; and/or
e.g., wherein the harvested clarified cell culture fluid (HOOF) is loaded onto the column downflow at about 6 cm/min; and/or
e.g., wherein HCCF loading is followed by an approximately 10 BV downflow wash; and/or
e.g., wherein the column is washed with J1 and J2, for example, wherein downflow wash is at about 6 cm/min with solution J1 and about 5 BV downflow wash at about 6 cm/min with solution J2 (pH 7.2, 10 mM sodium phosphate); and/or
e.g., wherein the column is eluted with solution J3 and the eluate is collected, for example, wherein after washing, downflow elution is carried out by a step gradient using about 10 BV of solution J3 at about 6 cm/min; and/or
e.g., wherein, during elution, when the online absorbance (at 280 nm wavelength; A₂₈₀) of the eluted peak reaches about 0.25 AU/cm, the collection of the pool is initiated and continued until the online absorbance (280 nm) drops below about 0.25 AU/cm; and/or
e.g., wherein the bed is sanitized for further use; and/or
e.g., wherein sanitization is via upflow at about 3 cm/min with about 3 BV of solution J4, about 3 BV of solution pH 4.5, 100 mM sodium acetate, and about 3 BV of solution of 20% (v/v) ethanol in pH 4.5, 100 mM sodium acetate; and/or
e.g., wherein, after sanitization, the bed is stored at 2-8° C.;
(2) inactivating viruses from the protein-A pooled eluate of step (1):
e.g., wherein viruses are inactivated by adjusting the antibody containing composition to about pH 3.5 for about 1 hour and then neutralized to about pH 5.5; and/or
e.g., wherein the protein-A eluate is adjusted to a pH of about 3.5; e.g., with solution J3 and, for example, held for about 1 hour; and/or
e.g., neutralizing the inactivated composition by adjusting the pH to about 5.5; and/or
e.g., wherein the pH is adjusted to about 5.5 with solution TRIS (1M tris(hydroxymethyl)aminomethane); and/or
e.g., wherein, after the virus is inactivated in the pool, filtering the inactivated pool through an about 0.45 micrometer and/or an about 0.2 micrometer filter (e.g., set up in series); and/or
e.g., wherein the inactivated and filtered pool is deposited into pre-sterilized vessels, such as bag(s); and/or
e.g., wherein, after the inactivated pool is filtered, the filters are washed; and/or
e.g., wherein the filters are washed with solution O1 (pH 5.5, 5 mM sodium acetate); and/or
e.g., in an embodiment of the invention, the inactivated and filtered pool is stored at about 2-8° C.; and
(3) purifying the inactivated pool on a strong cation-exchange chromatography column:
e.g., wherein the cation-exchange column medium comprises a sulfopropyl (—CH₂CH₂CH₂SO₃⁻) resin; and/or
e.g., wherein the cation-exchange resin comprises comprising a cross-linked poly(styrenedivinylbenzene) support matrix; and/or
e.g., wherein the resin matrix is packed at a bed height of about 20 cm before use; and/or
e.g., wherein the bed is equilibrated before loading with antibody with solution L1 (pH 5.5, mM sodium acetate, 20 mM sodium chloride); and/or
e.g., wherein column equilibration is upflow at about 5 cm/min for about 1 BV and downflow for about 11 BV with solution L1; and/or
e.g., wherein, after equilibrating the cation-exchange column, the antibody is loaded onto the column;
e.g., wherein the load of antibody per volume of packed resin is equal to or less than about 50 g/L; and/or
e.g., wherein the antibody solution is loaded onto the column downflow at about 5 cm/min; and/or
e.g., wherein, following loading of the antibody, the column is washed with solution L1; and/or
e.g., wherein, washing is with 10 BV downflow wash at about 5 cm/min with solution L1; and/or
e.g., wherein after washing the column is eluted with solution L2 (pH 5.5, 20 mM sodium acetate, 175 mM sodium chloride); and/or
e.g., wherein elution of the column comprises downflow eluting at about 5 cm/min; and/or
e.g., wherein elution is by a step gradient using about 15 BV of solution L2; and/or
e.g., wherein, during the elution step, when the online absorbance (280 nm) of the eluted peak reaches about 1.0 AU/cm, the collection of the pool is initiated and continued until about 4 BV is collected; and/or
e.g., wherein the pooled eluate is diluted with solution M1 (pH 8.0, 20 mM tris(hydroxymethyl)aminomethane hydrochloride); and/or
e.g., wherein, the eluted pool is diluted with solution M1, or with the solution used to equilibrate the next column in the purification scheme (e.g., anion-exchange column); and/or
e.g., wherein dilution, following elution, is by adding 2× the elution pool weight with solution M1 or the next column equilibration buffer, e.g., such that the conductivity of the pool is decreased to less than about 7.5 mS/cm; and/or
e.g., wherein the diluted pool is also adjusted to about pH 8; and/or
e.g., wherein the pH adjustment, to about 8, is with TRIS solution; and/or
e.g., wherein the eluted pool is filtered through a 0.2 micrometer pore size filter; and/or
e.g., wherein the cation-exchange resin bed is regenerated following elution; and/or
e.g., wherein the cation-exchange resin bed is regenerated with NaCl and NaOH; and/or
e.g., wherein the cation-exchange resin bed is regenerated downflow at about 2 cm/min with about 3 BV of 1M sodium chloride solution and, optionally, cleaned upflow at about 2 cm/min with about 5 BV of 0.5N sodium hydroxide solution and about 3 BV of 0.01 N sodium hydroxide solution; and
(4) purifying antibody on a strong anion-exchange chromatography column:
e.g., wherein the antibody does not bind the anion-exchange resin and washes through the column and elutes without application of an additional elution buffer; and/or
e.g., wherein the strong anion-exchange column resin is a quaternary ammonium; and/or
e.g., wherein the strong anion-exchange column resin matrix is a highly cross-linked agarose with dextran surface extender; and/or
e.g., wherein the column bed height is about 20 cm; and/or
e.g., wherein the column is equilibrated, before loading of the antibody, with solution M1; and/or
e.g., wherein the bed is equilibrated, before loading of the antibody, upflow at about 5 cm/min with about 1 BV and downflow with about 19 BV with solution M1; and/or
e.g., wherein the antibody is loaded onto the column after equilibration; for example, wherein the load of antibody per volume of packed resin is equal or less than about 50 g/L; and/or
e.g., wherein the antibody is loaded downflow onto the column at about 5 cm/min; and/or
e.g., wherein, following antibody loading, the column is washed with a wash buffer which washes the unbound antibody through the column; and/or
e.g., wherein, following antibody loading, the column is washed with solution M1; and/or
e.g., wherein the antibody load is followed by an approximately 5 BV wash downflow at about 5 cm/min with solution M1; and/or
e.g., wherein, during elution (of the antibody flow-through), when the online absorbance (280 nm) of the eluted peak reaches about 0.25 AU/cm, the collection of the pool is initiated and continued until the online absorbance (280 nm) drops below about 0.25 AU/cm; and/or
e.g., wherein the pool of collected eluate is adjusted to about pH 5.5; and/or
e.g., wherein the collected pool is adjusted to about pH 5.5 with solution J3; and/or
e.g., wherein, the eluate is filtered; and/or
e.g., wherein the eluate is filtered through a 0.1 micrometer pore size filter; and/or
e.g., wherein, following elution of the column, the column is regenerated; and/or
e.g., wherein the column is regenerated with NaCl and NaOH; and/or
e.g., wherein the column is regenerated downflow at about 2 cm/min with about 3 BV of 1M sodium chloride solution and cleaned upflow at about 2 cm/min with about 5 BV of 0.5N sodium hydroxide solution and about 3 BV of 0.01N sodium hydroxide solution; and
(5) filtering virus from the antibody:
e.g., wherein virus particles are filtered from the antibody by passing the antibody solution through a filter membrane comprising approximately 19 nm mean pore size; and/or
e.g., wherein the filter membrane used to filter the antibody solution has a capacity of about 300 L/m²; and/or
e.g., wherein, the filter is flushed, following filtration, of the antibody; and/or
e.g., wherein the filter is flushed, following filtration, with solution O1 (pH 5.5, 5 mM sodium acetate); and/or
e.g., wherein the solution O1 used to flush the filter after filtration is tested prior to use to ensure that it has an endotoxin level less than 1 EU/ml; wherein the filter is not used if the endotoxin level is 1 EU/ml or higher; and/or
e.g., wherein the filter is flushed until the absorbance (280 nm) of the permeate drops below about 0.1 AU/cm; and/or
e.g., wherein the antibody is filtered under pressure; and/or
e.g., wherein the filtering occurs under pressure, at a pressure drop of less than about 14 psi; and/or
e.g., wherein the filter is operated in the dead-end mode; and/or
e.g., wherein the integrity of the filter used is verified after completing the filtration; and/or
e.g., wherein, in an embodiment of the invention, the integrity of the viral filter is tested by the gold particle removal test as per the recommendation of the vendor; and/or
e.g., wherein, if the filter fails the integrity test after completing the filtration, the filtration step is repeated using an intact filter; and
(6) ultrafiltering and diafiltering the antibody solution:
e.g., wherein the antibody containing composition is ultrafiltered, then diafiltered; and/or
e.g., wherein the membranes used for filtration are initially flushed with solution O1; and/or
e.g., wherein the membranes used for filtration are initially flushed with 20 L/m² of solution O1; and/or
e.g., wherein the filter is initially flushed with solution O1 that is tested prior to use to ensure that it has an endotoxin level less than 1 EU/ml and not used if higher endotoxin levels are detected; and/or
e.g., wherein the filtrate generated during the virus filtration is concentrated, for example, by tangential flow ultrafiltration;
e.g., wherein, for example, the antibody is concentrated via ultrafiltration up to approximately 20 g/L; and/or
e.g., wherein, the antibody containing composition is diafiltered against solution O1; and/or
e.g., wherein the antibody is diafiltered against 10 diavolumes of solution O1; and/or
e.g., wherein the antibody is diafiltered for example, wherein the transmembrane pressure is maintained at about 15-25 psi; and/or
e.g., wherein the feed flow rate is about 6 L/min/m² during both concentration and/or diafiltration; and/or
e.g., wherein, following diafiltration and/or ultrafiltration, the retentate containing the antibody is concentrated further; and/or
e.g., wherein, following diafiltration and/or ultrafiltration, the antibody containing retentate is filtered through a 0.2 micrometer pore size filter; and/or
e.g., wherein the antibody is recovered from the diafiltration apparatus by flushing the system, e.g., with solution O1; and/or
e.g., wherein the diafiltration system is flushed with solution O1, for example, to obtain a concentration of the antibody in the range of about 35-50 g/L; and/or
e.g., wherein, after use, the ultrafiltration assembly is then cleaned; and/or
e.g., wherein the ultrafiltration assembly is cleaned with NaOH; and/or
e.g., wherein the ultrafiltration assembly is cleaned with 20 L/m² of 0.5N sodium hydroxide solution; and/or
e.g., wherein the ultrafiltration assembly is stored after use using about 20 L/m² of 0.01N sodium hydroxide solution; and
(7) performing a final filtration (fine filtration) of the antibody solution:
e.g., wherein the final filtration comprises passing the antibody containing solution through a 0.2 micron filter wherein the antibody is in the filtrate; and/or
e.g., wherein final filtration is into one or more pre-sterilized containers; and/or
e.g., wherein the final filtration operation is conducted in a laminar flow hood; and/or
e.g., wherein the containers into which the filtered material is placed are stored at about 2-8° C.; and/or
e.g., wherein the integrity of the filter used in the step is verified after completing the filtration; e.g., wherein, if the filter fails the integrity test, the step is repeated.

### Antibodies

The purification process of the present invention can be used to purify any protein, such as an antibody or antigen-binding fragment thereof, however, it is particularly useful for purifying an antibody that specifically binds IGF1R which antibody comprises immunoglobulin light chain F and immunoglobulin heavy chain A as set forth below.

19D12/15H12 Light Chain-F (LCF)
(SEQ ID NO: 1)

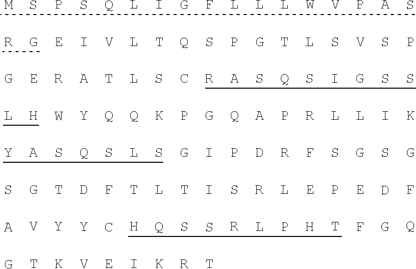

19D12/15H12 heavy chain-A (HCA)
(SEQ ID NO: 2)

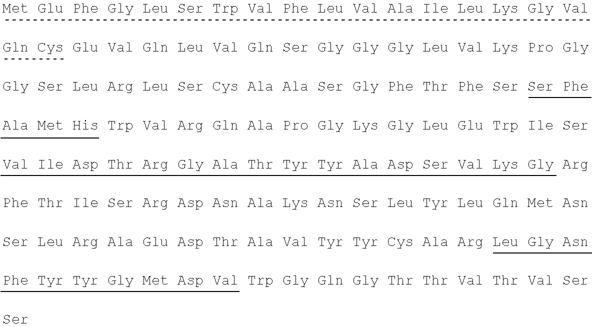

In the immunoglobulin sequences, the CDRs are underscored with a solid line and the signal sequence is underscored with a dashed line. The present invention includes methods for purifying an antibody or antigen-binding fragment thereof which comprises a mature light and/or heavy immunoglobulin variable region taken from SEQ ID NO: 1 and/or 2 or an immunoglobulin variable region the comprises 3 CDRs from SEQ ID NO: 1 and/or which comprises 3 CDRs from SEQ ID NO: 2, e.g., as defined by Kabat et al., Sequences of Proteins of Immunological Interest, 5^{th} Ed. Public Health Service, National Institutes of Health, Bethesda, Md. (1991); or, Chothia and Lesk, J. Mal. Biol. 196:901-917 (1987); or as specifically described above (underscored text indicating CDRs).

Purification of the antibodies and antigen-binding fragments thereof comprising the mature immunoglobulins or fragments thereof (e.g., comprising amino acid 20-128 of SEQ ID NO: 1 and/or comprising amino acids 20-137 of SEQ ID NO: 2), which lack the signal sequence, form part of the present invention.

In an embodiment of the invention, the light chain immunoglobulin variable region is fused to an immunoglobulin constant chain, e.g., a kappa chain. In an embodiment of the invention, the heavy chain immunoglobulin variable region is fused to an immunoglobulin constant chain, e.g., a gamma-1 chain.

### EXAMPLES

The following information is provided for more clearly describing the present invention and should not be construed to limit the present invention. Any and all of the compositions and methods described below, in whole or in part, fall within the scope of the present invention.

### Example 1

### Purification of Anti-IGF1R Antibody

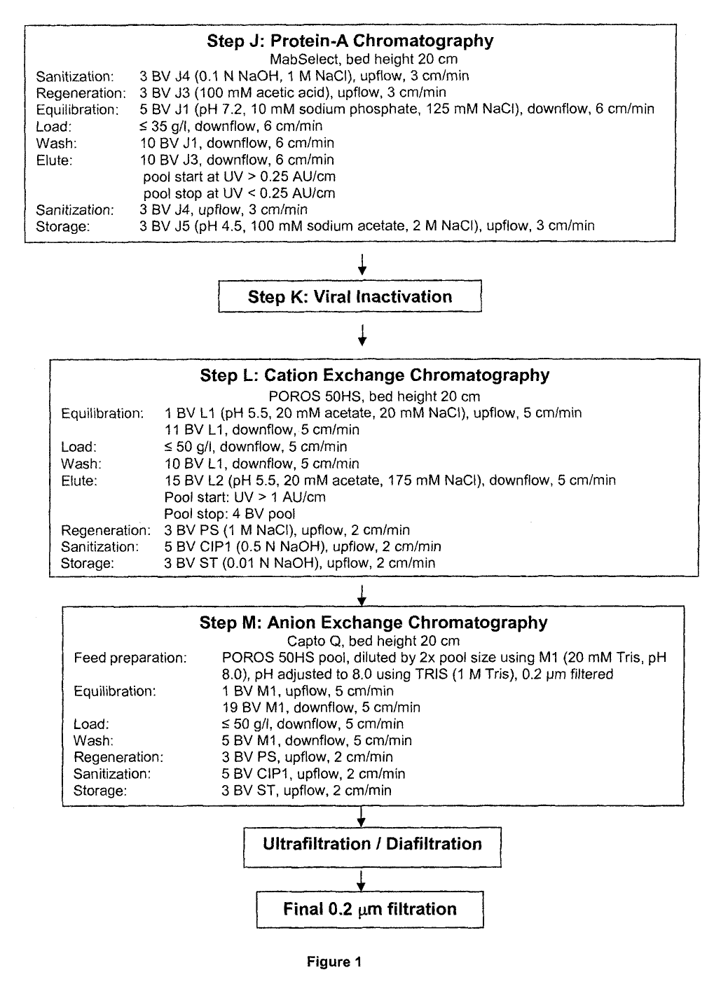

The process for the purification of anti-IGF1R (comprising amino acid 20-128 of SEQ ID NO: 1 and/or comprising amino acids 20-137 of SEQ ID NO: 2 (gamma-1/kappa)) included the following unit operations (see FIG. 1):

protein A chromatography;
viral inactivation;
cation-exchange chromatography;
anion-exchange chromatography;
virus filtration;
ultrafiltration/diafiltration (UF/DF); and
final filtration.

The purification procedure was carried out at room temperature (20-25° C.). The process delivered good quality antibody and removed aggregates, leached protein A, viruses, endotoxin, host cell proteins and DNA. At the same time, the process delivers appropriate control of bioburden in process intermediates and purified antibody.

The chromatography resins used were:

MabSelect (GE Healthcare; Piscataway, N.J.; Catalog number 17-5199)-Protein-A chromatography resin;
POROS 50HS (Applied Biosystems; Foster City, Calif.; Cat. no. 1-3359)-Cation-exchange chromatography resin;
Capto Q (GE Healthcare, Cat. no. 175316)-Anion-exchange chromatography resin.

The filters used were:

Millistak+ A1HC (Millipore; Billerica, Mass.) depth filter;
Planova 20N virus filter (Asahi Kasei; and
Pellicon 2 cassettes with Biomax-50 A membranes (Millipore).

Preparation of Equipment and Solutions.

The solutions used were made using WFI (water for injection) and sterile filtered using 0.2 micrometer filters. Samples of diafiltration solution were assayed for endotoxin by Limulus amebocyte lysate (LAL) tests and only those with <0.3 EU/ml were used for the purification. The chromatography system was washed in 0.5 N NaOH and held for an hour in order to minimize the presence of endotoxin and bioburden.

Protein A Chromatography (Step J).

Protein-A served as a capture and initial purification step for the antibody. The chromatography media used in this step (MabSelect) was highly specific for human IgG1 (such as anti-IGF1R). The binding occurred between the constant region (Fc) of the antibody and the ligand (Protein-A). Accordingly, the mAb bound to the column and other contaminants present in the HCCF that exhibited much less affinity for the Protein-A ligand (e.g. host cell proteins and DNA) flowed through.

Mabselect chromatography media (GE Healthcare, Piscataway, N.J., USA) was packed at a bed height of 20 cm. The bed was sanitized upflow at 3 cm/min with 3 bed volumes (BV) of solution J4 (0.1N sodium hydroxide, 1M sodium chloride), regenerated upflow at 3 cm/min with 3 BV of solution J3 (100 mM acetic acid, pH 2.9) and equilibrated downflow at 6 cm/min with 5 BV of solution J1 (pH 7.2, 10 mM sodium phosphate, 125 mM sodium chloride).

The load of mAb per volume of packed resin was about 35 g/L. The harvested clarified culture medium (HCCM) was loaded onto the column downflow at 6 cm/min, followed by a 10 BV downflow wash at 6 cm/min with solution J1 and 5 BV downflow wash at 6 cm/min with solution J2 (pH 7.2, 10 mM sodium phosphate). After washing, downflow elution was carried out by a step gradient using 10 BV of solution J3 at 6 cm/min. When the online absorbance (280 nm) of the eluted peak reached 0.25 AU/cm, the collection of the pool was initiated and continued until the online absorbance (280 nm) dropped below 0.25 AU/cm.

The bed was sanitized upflow at 3 cm/min with 3 BV of solution J4 and 3 BV of solution pH 4.5, 100 mM sodium acetate, and 3 BV of solution of 20% (v/v) ethanol in pH 4.5, 100 mM sodium acetate (PROA ST). The bed was stored at 2-8° C.

Viral Inactivation (Step K).

The goal of this step was to carry out the inactivation of certain viruses that might be present in the pool. The pool collected during the Protein-A chromatography step was adjusted immediately to pH 3.5 with solution J3 and held for 1 hour. Afterwards, the pH of the inactivated pool was adjusted to 5.5 with solution TRIS (1M tris(hydroxymethyl)aminomethane). The inactivated pool was then filtered through 0.45 micrometer and 0.2 micrometer filters (set up in series) into pre-sterilized bag(s) and the filters were washed with solution O1 (pH 5.5, 5 mM sodium acetate). This intermediate was then stored at 2-8° C.

Cation-Exchange Chromatography (Step L).

During this step, the concentrations of impurities present in the feed, such as aggregates, Protein-A (co-eluted with mAb during the Protein-A chromatography step), host cell proteins and DNA, were reduced, thus affecting purification.

POROS 50 HS chromatography media (Applied Biosystems, Foster City, Calif., USA) was packed at a bed height of 20 cm. The bed was equilibrated upflow at 5 cm/min for 1 BV and downflow for 11 BV with solution L1 (pH 5.5, 20 mM sodium acetate, 20 mM sodium chloride).

The load of mAb per volume of packed resin was about 50 g/L. The viral-inactivated intermediate from step K was loaded onto the column downflow at 5 cm/min, followed by a 10 BV downflow wash at 5 cm/min with solution L1. After washing, downflow elution was carried out at 5 cm/min by a step gradient using 15 BV of solution L2 (pH 5.5, 20 mM sodium acetate, 175 mM sodium chloride). When the online absorbance (280 nm) of the eluted peak reached 1.0 AU/cm, the collection of the pool was initiated and continued until 4 BV were collected. The pool was diluted with solution M1 (pH 8.0, 20 mM tris(hydroxymethyl)aminomethane hydrochloride) by adding an amount of M1 equal to 2 times the pool weight. In this fashion, the conductivity of the pool was decreased to less than 7.5 mS/cm. After dilution, the pool was adjusted to pH 8.0 with solution TRIS and 0.2 micrometer filtered.

The bed was regenerated downflow at 2 cm/min with 3 BV of 1M sodium chloride solution and cleaned upflow at 2 cm/min with 5 BV of 0.5N sodium hydroxide solution and 3 BV of 0.01N sodium hydroxide solution.

Anion-Exchange Chromatography (Step M).

During this step the mAb did not bind to the column since its pI was higher than the pH at which the chromatography was run. Negatively-charged contaminants present in the feed, such as viruses, DNA or host cell proteins, bound to the resin, thus effecting purification.

Capto Q chromatography media (GE Healthcare, Piscataway, N.J., USA) was packed at a bed height of 20 cm. The bed was equilibrated upflow at 5 cm/min for 1 BV and downflow for 19 BV with solution M1.

The load of mAb per volume of packed resin was about 45 g/L. The adjusted pool from step L was loaded downflow onto the column at 5 cm/min, followed by a 5 BV wash downflow at 5 cm/min with solution M1. When the online absorbance (280 nm) of the eluted peak reached 0.25 AU/cm, the collection of the pool was initiated and continued until the online absorbance (280 nm) dropped below 0.25 AU/cm. The collected pool was adjusted to pH 5.5 with solution J3 and 0.1 micrometer filtered.

The bed was regenerated downflow at 2 cm/min with 3 BV of 1M sodium chloride solution and cleaned upflow at 2 cm/min with 5 BV of 0.5N sodium hydroxide solution and 3 BV of 0.01N sodium hydroxide solution.

Virus Filtration.

During this step, small viruses that might have been present in the pool were retained by the filter (19 nm mean pore size) while the mAb flowed through the membrane.

A Planova 20N filter (Asahi Kasei Pharma; Tokyo, Japan) was loaded at a capacity of about 80 L/m² during this step. The filter was flushed with solution O1. Solution O1 was tested prior to use to ensure that it has an endotoxin level less than 1 EU/ml.

The adjusted pool from step M was filtered at a pressure drop of approximately 12 psi. The filter was operated in the dead-end mode. Once all the pool volume was fed to the filter, the device was flushed with solution O1 until the absorbance (280 nm) of the permeate dropped below 0.1 AU/cm.

The integrity of the filter used in the step was verified after completing the filtration. The filter passed the integrity test. If the filter had failed the integrity test, the step would have been repeated.

Ultrafiltration/Diafiltration.

During this step, the mAb was retained by the membrane (in the retentate). A goal of this step was to adjust the mAb concentration, solution composition, pH and conductivity of the active pharmaceutical ingredient (API; antibody) composite.

Pellicon-2 cassettes with A-screens (Millipore Corporation, Billerica, Mass.) and Biomax 50 membranes (50 kD nominal molecular weight cutoff) were assembled using an appropriate holder. The assembly was then flushed with 20 L/m² of solution O1. Solution O1 was tested prior to use to ensure that it has an endotoxin level less than 1 EU/ml.

The filtrate generated during the virus filtration was concentrated by tangential flow ultrafiltration up to approximately 20 g/L. After concentration, the retentate was diafiltered against 10 diavolumes of solution O1. The transmembrane pressure was maintained at 19.5-20 psi and the feed flow rate at about 6 L/min/m² during both concentration and diafiltration. Following diafiltration, the retentate was concentrated further, 0.2 micrometer filtered and the mAb was recovered by flushing the system with solution O1 to obtain a concentration of the antibody drug substance in the range of about 44 g/L.

The ultrafiltration assembly was cleaned with 20 L/m² of 0.5N sodium hydroxide solution and stored using 20 L/m² of 0.01N sodium hydroxide solution.

0.2 Micrometer Final Filtration (Fine Filtering).

The API composite generated during the ultrafiltration was 0.2 micrometer filtered into pre-sterilized containers. The operation was conducted in a laminar flow hood. The containers were stored at 2-8° C. in a refrigerator. The integrity of the filter used in the step was verified after completing the filtration. The filter passed the integrity test. If the filter had failed the integrity test, the step would have been repeated.

Tables 7 and 8 set forth the anti-IGF1R purity and LRV profile typically obtained using the purification process of the present invention.

TABLE 7
Typical Purity Results of the Drug Substance Generated by the
Purification Process

| Test | Results |
|---|---|
| Physical observation | Turbid, contained particulates. |
| | (Comment: observed |
| | nephelometric turbidity is 3-6 NTU) |
| pH | 5.8 |
| Assay UV | 44.1 mg/mL |
| Biological potency assay | 1.2 × 10⁵ SPU/mg |
| KIRA (kinase receptor activation | |
| assay) | |
| Purity | 0.9% Total Impurities |
| SDS-PAGE (Reducing) | |
| Purity | 13.3% Total Impurities |
| SDS-PAGE (Non-reducing) | |
| Purity | Monomer: 99.3% |
| HPSEC (high pressure size | High Molecular Weight Species: |
| exclusion chromatography) | < QL (QL of 0.38%) |
| | Late Eluting Peaks: 0.38% |
| | (QL of 0.38%) |
| Identification | Positive |
| KIRA | |
| Purity | 2 PPM (QL: 1 PPM) |
| Residual Host Cell Protein (CHO) | |
| Purity | < QL (QL = 20 pg/4 mg) |
| Residual Host Cell DNA (CHO) | |
| Purity | Residual Protein A not detected |
| Residual Protein A | (QL: 2 ng Protein A/mL) |
| Bacterial Endotoxin | <0.01 EU/mg |
| LAL | |
| Isoelectric Focusing | Total bands: 3 (pH 8.3-9.5) |
| Charged Variants | No visual difference to Ref. Std. |

TABLE 8
Typical Virus log reduction values (LRV) for key steps in the
purification process

| | LRV | |
|---|---|---|
| | MMV | XMuLV |
| | (Mouse minute | (xenotropic murine |
| Step | virus) | leukemia virus) |
| Viral inactivation | NA | ≧6.71 ± 0.25 |
| Anion-exchange chromatography | 5.63 ± 1.07 | ≧4.00 ± 0.26 |
| Virus filtration | ≧5.33 ± 0.40 | ≧5.85 ± 0.35 |

Table 9 sets forth the results of antibody purification using the various resins and conditions set forth therein.

TABLE 9
Results from Step Gradient Elution on different Cation Exchangers at 50 g/l Load

| | | | Pool Purity | | | | | |
|---|---|---|---|---|---|---|---|---|
| | | | | HCP | DNA | Pro-A | | Pool |
| | Step elution | Flow rate | Aggregate | (ng/mg | (ng/mg | (ng/mg | Yield | Size |
| Resins | condition | (cm/min) | (%) | IgG) | IgG) | IgG) | (%) | (BV) |
| Cation | NA | NA | 4.46 | 5.87 ± 0.1 | 5.9 | 3 | NA | NA |
| exchange | | | | | | | | |
| feed | | | | | | | | |
| FractogelSE | 20 mM acetate, | 2 | 0.28 | 1.22 ± 0.07 | 2.1 | <3 | 77 | 4 |
| Hicap | 175 mM NaCl, | | | | | | | |
| | pH 5.5. | | | | | | | |
| FractogelSE | 20 mM acetate, | 5 | 0.35 | 1 | 12.7 | <3 | 84 | 3 |
| Hicap | 175 mM NaCl, | | | | | | | |
| | pH 5.5. | | | | | | | |
| POROS | 20 mM acetate, | 5 | 0.23 | 1.48 ± 0.06 | 1.6 | <3 | 74 | 4 |
| 50HS | 175 mM NaCl, | | | | | | | |
| | pH 5.5. | | | | | | | |
| SP | 20 mM acetate, | 2 | 0.38 | 7 | 64.2 | <3 | 75 | 12 |
| Sepharose | 140 mM NaCl, | | | | | | | |
| FF | pH 5.5. | | | | | | | |
| SP 550C | 20 mM acetate, | 2 | 0.80 | 2.31 ± 0.01 | 11.3 | <3 | 82 | 7 |
| | 200 mM NaCl, | | | | | | | |
| | pH 5.5. | | | | | | | |
| UNOSphere S | 20 mM acetate, | 2 | 0.48 | 3.27 ± 0.01 | 6.6 | <3 | 81 | 6 |
| | 220 mM NaCl, | | | | | | | |
| | pH 5.5. | | | | | | | |

HCP = host cell proteins

Table 10 sets forth the purity profile of antibody isolated by a particular run of the purification process set forth in this example.

TABLE 10
Specific Purity Results of the Drug Substance Generated by a Single
Run of the Purification Process

| Test | Results |
|---|---|
| HPSEC (High performance size | Monomer: 99.3% |
| exclusion chromatography) | Aggregates: <0.38% |
| Residual Host Cell Protein | 2 ng HCP/mg mAb |
| (CHO) | |
| Residual Host Cell DNA (CHO) | <5 pg DNA/mg mAb |
| Residual Protein A | :<0.05 ng Protein A/mg mAb |
| Bacterial Endotoxin (LAL) | <0.01 EU/mg mAb |
| Bioburden | 0 CFU/mL |

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A method for purifying an antibody or antigen-binding fragment thereof in an aqueous composition that specifically binds IGF1R which comprises a CDR-L1, CDR-L2 and CDR-L3 found in a light chain immunoglobulin variable region which comprises the amino acid sequence set forth in SEQ ID NO: 1; and a CDR-H1, CDR-H2 and CDR-H3 found in a heavy chain immunoglobulin variable region which comprises the amino acid sequence set forth in SEQ ID NO: 2; which method comprises:
(a) purifying the antibody by protein A chromatography;
(b) inactivating virus particles in the composition;
(c) purifying the antibody by cation-exchange chromatography;
(d) purifying the antibody anion-exchange chromatography;
(e) filtering virus particles from the composition;
(f) ultrafiltering the composition;
(g) diafiltering the composition; and
(h) fine filtering the composition.

2. The method of claim 1 wherein the purification is performed between about 20° C. and 25° C.

3. The method of claim 1 wherein protein A chromatography comprises:
applying harvested cell culture fluid, comprising the antibody or fragment, to a column comprising a protein-A/agarose resin equilibrated with an aqueous solution comprising 10 mM sodium phosphate, 125 mM sodium chloride pH 7.2; wherein the ratio of antibody to volume of resin is about 35 grams/liter;
washing the column with an aqueous solution comprising 10 mM sodium phosphate, 125 mM sodium chloride pH 7.2;
washing the column with an aqueous solution comprising 10 mM sodium phosphate, pH 7.2;
eluting the antibody from the resin with 100 mM acetic acid, pH 2.9; and
collecting the antibody in the eluate.

4. The method of claim 1 wherein the virus particles are inactivated by adjusting the pH of the composition to about 3.5 for about 1 hour.

5. The method of claim 4 wherein the pH is adjusted to about 5.5 after the virus particles are inactivated.

6. The method of claim 1 wherein cation-exchange chromatography comprises:
applying the antibody or fragment to a column comprising —CH₂CH₂CH₂SO₃⁻ strong cation-exchange resin equilibrated with an aqueous solution comprising 20 mM sodium acetate, 20 mM sodium chloride, pH 5.5;
washing the column with an aqueous solution comprising 20 mM sodium acetate, 20 mM sodium chloride, pH 5.5;
eluting the antibody or fragment from the resin with an aqueous solution comprising 20 mM sodium acetate, 175 mM sodium chloride, pH 5.5; and
collecting antibody or fragment containing eluate starting when the A₂₈₀ of the eluate reaches about 1.0 AU/cm and finishing when about 4 bed volumes have been collected.

7. The method of claim 1 wherein anion-exchange chromatography comprises:
applying the antibody or fragment to a column comprising strong quaternary ammonium (Q) anion-exchanger resin equilibrated with an aqueous solution comprising 20 mM tris(hydroxymethyl)aminomethane hydrochloride, pH 8.0;
washing the column with an aqueous solution comprising 20 mM tris(hydroxymethyl)aminomethane hydrochloride, pH 8.0, wherein the solution washes unbound antibody or fragment through the column; and
collecting the antibody or fragment in the washed-through eluate.

8. The method of claim 1 wherein ultrafiltering the antibody or fragment comprises filtering the antibody or fragment through a membrane under pressure of about 15-25 pounds per square inch (psi) by tangential flow.

9. The method of claim 1 wherein diafiltering the antibody or fragment comprises filtering the antibody or fragment through a membrane under pressure of about 15-25 pounds per square inch (psi) against about 10 volumes of 5 mM sodium acetate, pH 5.5.

10. The method of claim 1 wherein fine filtering the antibody or fragment comprises filtering the antibody or fragment through a filter with a pore size of about 0.2 micrometers.

11. A method of claim 1 wherein the antibody or fragment is initially in a harvested clarified culture medium produced by a method comprising:
inoculating an initial mammalian cell growth medium with host cells expressing the antibody or fragment and adding supplements comprising:
Glucose;
L-glutamine;
Soy hydrolysate or wheat hydrolysate or both;
Adenine sulfate;
Adenosine;
ammonium vanadate;
Biotin;
Choline Chloride;
Cobalt chloride;
Cupric sulfate;
Cytidine;
D-Calcium Pantothenate;
Ethanolamine HCl;
Flavin Adenine Dinucleotide;
Folic Acid;
Glycine;
Guanosine;
Hypoxanthine;
i-Inositol;
L-alanine;
L-arginine;
L-asparagine;
L-aspartic acid;
L-citrulline;
L-cysteine-HCl;
L-cystine;
L-glutamic acid;
L-histidine;
Lipoic Acid;
L-isoleucine;
L-leucine;
L-lysine;
L-methionine;
L-ornithine-HCl;
L-phenylalanine;
L-proline;
L-serine;
L-threonine;
L-tryptophan;
L-tyrosine;
L-valine;
Manganese chloride Tetrahydrate;
Niacin;
Nickel dichloride hexahydrate;
Progesterone;
Putrescine 2HCl;
Pyridoxine HCl;
Riboflavin;
Sodium molybdate dehydrate;
Sodium phosphate monobasic;
Sodium selenite;
Thiamine HCl;
Thymidine;
Tin chloride dehydrate;
Uridine;
Vitamin B12;
Vitamin E; and
Zinc sulfate;
to the medium; and removing the antibody and culture medium from the host cells.

12. The method of claim 11 wherein the final concentrations of the components added to the medium from the supplements are about those set forth below:

| | | |
|---|---|---|
| Adenine sulfate: | 1.632 | mg/liter |
| Adenosine: | 17.6 | mg/liter |
| Ammonium vanadate: | 0.00078 | mg/liter |
| Biotin: | 0.28 | mg/liter |
| Choline Chloride: | 50.2 | mg/liter |
| Cobalt chloride: | 0.0025 | mg/liter |
| Cupric sulfate: | 0.0032 | mg/liter |
| Cytidine: | 17.6 | mg/liter |
| D-Calcium Pantothenate: | 23.8 | mg/liter |
| Ethanolamine HCl: | 4.4 | mg/liter |
| Flavin Adenine Dinucleotide: | 0.05 | mg/liter |
| Folic Acid: | 4.6 | mg/liter |
| Glycine | 72 | mg/liter |
| Guanosine: | 17.6 | mg/liter |
| Hypoxanthine: | 11.8 | mg/liter |
| i-Inositol: | 73.2 | mg/liter |
| L-alanine: | 8.9 | mg/liter |
| L-arginine | 312.4 | mg/liter |
| L-asparagine: | 842 | mg/liter |
| L-aspartic acid | 97.6 | mg/liter |
| L-citrulline: | 12.6 | mg/liter |
| L-cysteine-HCl | 224 | mg/liter |
| L-cystine: | 34 | mg/liter |
| L-glutamic acid | 155.4 | mg/liter |
| L-histidine | 167 | mg/liter |
| Lipoic Acid: | 0.52 | mg/liter |
| L-isoleucine | 422 | mg/liter |
| L-leucine | 384 | mg/liter |
| L-lysine | 365 | mg/liter |
| L-methionine | 147.2 | mg/liter |
| L-ornithine-HCl: | 25.6 | mg/liter |
| L-phenylalanine | 207 | mg/liter |
| L-proline | 239 | mg/liter |
| L-serine: | 281 | mg/liter |
| L-threonine | 211.6 | mg/liter |
| L-tryptophan | 109.2 | mg/liter |
| L-tyrosine | 234 | mg/liter |
| L-valine | 308.8 | mg/liter |
| Manganese chloride tetrahydrate: | 0.0003 | mg/liter |
| Niacin: | 31.4 | mg/liter |
| Nickel dichloride hexahydrate: | 0.0004 | mg/liter |
| Progesterone: | 0.015 | mg/liter |
| Putrescine 2HCl: | 0.4 | mg/liter |
| Pyridoxine HCl: | 3 | mg/liter |
| Riboflavin: | 1.86 | mg/liter |
| Sodium molybdate dehydrate: | 0.00016 | mg/liter |
| Sodium phosphate monobasic: | 288.2 | mg/liter |
| Sodium selenite: | 0.01426 | mg/liter |
| Thiamine HCl: | 16 | mg/liter |
| Thymidine: | 7.8 | mg/liter |
| Tin chloride dehydrate: | 0.00008 | mg/liter |
| Uridine: | 17.6 | mg/liter |
| Vitamin B12: | 3.4 | mg/liter |
| Vitamin E: | 0.376 | mg/liter |
| Zinc sulfate: | 1.08 | mg/liter |
| Glucose | 1.5 | g/liter |
| L-glutamine | 150 | mg/liter |

13. The method of claim 11 wherein the supplements are added from an amino acid feed that comprises amino acids at about the following concentrations:
L-arginine: 6.32 g/liter
L-cystine: 1.7 g/liter
L-histidine 2.1 g/liter
L-isoleucine: 2.6 g/liter
L-leucine: 2.6 g/liter
L-lysine: 3.6 g/liter
L-Methionine: 0.76 g/liter
L-phenylalanine: 1.65 g/liter
L-threonine: 2.38 g/liter
L-tryptophan: 0.51 g/liter
L-tyrosine: 1.8 g/liter
L-valine: 2.34 g/liter

14. The method of claim 13 wherein about 20 ml amino acid feed is added per liter of culture medium.

15. The method of claim 11 wherein the supplements are added from an amino acid feed that comprises amino acids at about the following concentrations:
L-alanine: 0.89 g/liter
L-asparagine: 1.5 g/liter
L-aspartic acid: 1.33 g/liter
L-glutamic acid: 1.47 g/liter
Glycine: 0.75 g/liter
L-proline: 1.15 g/liter
L-serine: 1.05 g/liter

16. The method of claim 15 wherein about 10 ml amino acid feed is added per liter of culture medium.

17. The method of claim 11 wherein the supplements are added from a nutrient feed that comprises supplements at about the following concentrations:

| | | |
|---|---|---|
| L-asparagine: | 40.6 | g/liter |
| L-proline | 10.81 | g/liter |
| L-isoleucine | 18.53 | g/liter |
| L-cysteine-HCl | 11.19 | g/liter |
| L-leucine | 16.58 | g/liter |
| L-threonine | 8.2 | g/liter |
| L-tyrosine | 9.9 | g/liter |
| L-arginine | 9.29 | g/liter |
| L-aspartic acid | 3.56 | g/liter |
| L-glutamic acid | 6.28 | g/liter |
| Glycine | 2.83 | g/liter |
| L-histidine | 6.23 | g/liter |
| L-methionine | 6.58 | g/liter |
| L-tryptophan | 4.93 | g/liter |
| L-lysine | 14.66 | g/liter |
| L-phenylalanine | 8.64 | g/liter |
| L-valine | 13.08 | g/liter |
| L-serine: | 13 | g/liter |
| Sodium phosphate Monobasic: | 14.41 | g/liter |
| Zinc sulfate: | 0.054 | g/liter |
| Cupric sulfate: | 0.00016 | g/liter |
| Ammonium vanadate: | 0.000039 | g/liter |
| Cobalt chloride: | 0.000125 | g/liter |
| Nickel dichloride Hexahydrate: | 0.00002 | g/liter |
| Sodium molybdate dehydrate: | 0.000008 | g/liter |
| Tin chloride dehydrate: | 0.000004 | g/liter |
| Manganese chloride: tetrahydrate: | 0.000015 | g/liter. |

18. The method of claim 17 wherein about 20 ml nutrient feed is added per liter of culture medium.

19. The method of claim 11 wherein the supplements are added from a vitamin/salt feed that comprises supplements at about the following concentrations:
Sodium selenite: 7.13×10⁻⁴ g/liter
Adenine sulfate: 0.0816 g/liter
Adenosine: 0.88 g/liter
Cytidine: 0.88 g/liter
Guanosine: 0.88 g/liter
Uridine: 0.88 g/liter
Hypoxanthine: 0.59 g/liter
L-citrulline: 0.63 g/liter
L-ornithine-HCl: 1.28 g/liter
Biotin: 0.014 g/liter
Flavin Adenine Dinucleotide: 0.0025 g/liter
Folic Acid: 0.23 g/liter
Lipoid Acid: 0.026 g/liter
Niacin: 1.57 g/liter
Pyridoxine HCl: 0.15 g/liter
Riboflavin: 0.093 g/liter
Thiamine HCl: 0.8 g/liter
Vitamin E: 0.0188 g/liter
Vitamin B12: 0.17 g/liter
Choline Chloride: 2.51 g/liter
Ethanolamine HCl: 0.22 g/liter
i-Inositol: 3.66 g/liter
Thymidine: 0.39 g/liter
Putrescine 2HCl: 0.02 g/liter
Progesterone: 0.00075 g/liter
D-Calcium Pantothenate: 1.19 g/liter

20. The method of claim 19 wherein about 20 ml vitamin/salt feed is added per liter of culture medium.

21. The method of claim 11 wherein the culture medium is harvested from the host cells when viability of the cells is below about 60%.

22. The method of claim 11 further comprising purifying the antibody or fragment and culture medium from the cells by centrifuging the antibody or fragment and culture medium and/or depth filtering the antibody or fragment and culture medium and/or filtering the antibody or fragment and culture medium through a 0.2 micron filter.

23. The method of claim 11 wherein the initial mammalian cell growth medium to which the supplements are added comprises: HEPES, sodium bicarbonate buffers, inorganic salts, non-essential amino acids, recombinant human insulin, trace elements and surfactants; and does not comprise L-glutamine, antibiotics, antimycotics or animal-derived components.

24. The method of claim 11 comprising: inoculating an initial mammalian cell growth medium, pre-warmed to about 37° C.; which initial medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing the light chain immunoglobulin and heavy chain immunoglobulin, to a cell density of about 2.5-5×10⁵ cells/ml; and, adding the following supplements to the medium before, simultaneously with or immediately after said inoculation:
soy hydrolysate to a final concentration of about 10 g/liter;
and, optionally, an amino acid feed wherein the concentrations of the components added by said amino acid feed are approximately those set forth below:
L-arginine: 126.4 mg/liter
L-cystine: 34 mg/liter
L-histidine: 42 mg/liter
L-isoleucine: 52 mg/liter
L-leucine: 52 mg/liter
L-lysine: 72 mg/liter
L-Methionine: 15.2 mg/liter
L-phenylalanine: 33 mg/liter
L-threonine: 47.6 mg/liter
L-tryptophan: 10.2 mg/liter
L-tyrosine: 36 mg/liter
L-valine: 46.8 mg/liter
L-alanine: 8.9 mg/liter
L-asparagine: 30 mg/liter
L-aspartic acid: 26.6 mg/liter
L-glutamic acid: 29.4 mg/liter
glycine: 15 mg/liter
L-proline: 23 mg/liter
L-serine: 21 mg/liter;
and, when viable cell density reaches over about 1.2×10⁶ cells/ml, adding supplement feeds wherein the concentrations of the components added by said supplement feeds are approximately those set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, maintaining glucose concentration in the medium at about 1.5 g/liter and maintaining L-glutamine concentration in the medium at about 150 mg/liter; and during cell growth maintaining O₂ concentration at about 60%; pH at about 6.8±0.02 and temperature at about 36.5° C.±0.5° C.; and, optionally, removing the host cells from the medium when cell viability is below about 60%.

25. The method of claim 24 wherein host cells are removed from the medium by a method comprising disk-stack centrifuging the medium, depth filtering the medium and filtering the medium through a filter with about a 0.2 micron pore size.
